(19) United States
(12) Patent Application Publication
(10) Pub. No.: US 2025/0145981 A1
(43) Pub. Date: May 8, 2025

(54) CHEMOENZYMATIC CARBOXYLATION COMPOSITIONS AND METHODS

(71) Applicant: Solugen, Inc., Houston, TX (US)

(72) Inventors: Toni M. Lee, Missouri City, TX (US); Konrad V. Miller, Sugar Land, TX (US); Gaurab Chakrabarti, Houston, TX (US); Sean Hunt, Houston, TX (US); Camille Boucher-Jacobs, Houston, TX (US); Hans-Joerg Woelk, Hamburg (DE)

(73) Assignee: Solugen, Inc., Houston, TX (US)

(21) Appl. No.: 18/939,461

(22) Filed: Nov. 6, 2024

Related U.S. Application Data
(60) Provisional application No. 63/596,312, filed on Nov. 6, 2023.

(51) Int. Cl. C12N 9/88

(52) U.S. Cl. CPC C12N 9/88; C12Y 401/01001

(57) ABSTRACT

A method of increasing the carbon content of an organic compound comprising carboxylating an organic compound characterized by the general formula R¹—H and carbon dioxide in the presence of a biocatalyst under conditions suitable for the formation of a carboxylic acid characterized by the general formula R¹—OOH, wherein R¹ is a C₁ to C₃₀ organyl group; a C₁ to C₃₀ hydrocarbyl group, a C₃ to C₃₀ aromatic group; a C₁ to C₃₀ alkyl group, a C₄ to C₃₀ cycloalkyl group, a C₄ to C₃₀ substituted cycloalkyl group, a C₃ to C₃₀ aliphatic heterocyclic group, a C₃ to C₃₀ substituted aliphatic heterocyclic group, a C₆ to C₃₀ aryl group, a C₆ to C₃₀ substituted aryl group, a C₃ to C₃₀ heteroaryl group, or a C₃ to C₃₀ substituted heteroaryl group or combinations thereof.

### CROSS-REFERENCE TO RELATED APPLICATIONS

This application claims priority to U.S. Provisional Application Ser. No. 63/596,312 filed Nov. 6, 2023 entitled “CHEMOENZYMATIC CARBOXYLATION COMPOSITIONS AND METHODS,” which is incorporated herein by reference in its entirety for all purposes.

### STATEMENT REGARDING FEDERALLY SPONSORED RESEARCH OR DEVELOPMENT

Not applicable.

### FIELD

The present disclosure relates generally to carbon fixation. More particularly, this disclosure relates to chemoenzymatic carboxylation compositions and methods.

### BACKGROUND

Over the last 150 years, synthesis of inexpensive chemicals from fossilized forms of carbon (e.g. oil, coal, natural gas) has dramatically altered society through their broad applications, ranging from cosmetics to plastics. This petroleum-based carbon feedstock generates a small collection of platform chemicals from which highly efficient chemical conversions lead to the manufacture of a large variety of chemical products. However, the current approach to producing these carbon-based chemicals is inherently non-sustainable as feedstocks that required millions of years to form are being depleted. The creation of a truly sustainable chemical industry will only occur when the timescale of the feedstock formation matches the timescale of its utilization to make chemicals.

Only a few ‘chemical carbon fixation’ processes are performed on an industrial scale. Urea synthesis is performed by combining H₂ and N₂ gas to generate ammonia which is then reacted with CO₂ to form urea, a process that must operate under harsh conditions and consumes more than 2% of the world's energy. Microbial carbon fixation systems for generating chemicals with increased carbon count have largely focused on using algae as a host, but suffer from technoeconomically prohibitive low yields and difficult downstream processing steps.

Dwindling petroleum feedstocks and increased CO₂-concentrations in the atmosphere have rendered the concept of using CO₂ as a raw material for synthesis of chemical compounds attractive. Having the highest oxidation state possible for carbon, CO₂ has a low energy level and thus a large energy input is required for its transformation. Thus, an ongoing need exists for developing methods for carbon fixation that lack one or more of the challenges associated with the current methodologies.

### SUMMARY

A method of increasing the carbon content of an organic compound comprising carboxylating an organic compound characterized by the general formula R¹—H and carbon dioxide in the presence of a biocatalyst under conditions suitable for the formation of a carboxylic acid characterized by the general formula R¹—OOH, wherein R¹ is a C₁ to C₃₀ organyl group; a C₁ to C₃₀ hydrocarbyl group, a C₃ to C₃₀ aromatic group; a C₁ to C₃₀ alkyl group, a C₄ to C₃₀ cycloalkyl group, a C₄ to C₃₀ substituted cycloalkyl group, a C₃ to C₃₀ aliphatic heterocyclic group, a C₃ to C₃₀ substituted aliphatic heterocyclic group, a C₆ to C₃₀ aryl group, a C₆ to C₃₀ substituted aryl group, a C₃ to C₃₀ heteroaryl group, or a C₃ to C₃₀ substituted heteroaryl group or combinations thereof. FIG. 1 a schematic of a carboxylase reaction.

### BRIEF DESCRIPTION OF DRAWINGS

For a detailed description of various exemplary embodiments, reference will now be made to the accompanying drawings in which:

FIG. 1 is a schematic depiction of a carboxylase reaction;

FIG. 2 is a schematic of a chemoenzymatic carboxylation reaction; and

FIG. 3 is an aspect of a process flow diagram of a chemoenzymatic process of the type disclosed herein.

### DETAILED DESCRIPTION

The following discussion is directed to various exemplary aspects. However, one of ordinary skill in the art will understand that the examples disclosed herein have broad application, and that the discussion of any aspect is meant only to be exemplary of that aspect, and not intended to suggest that the scope of the disclosure, including the claims, is limited to that aspect.

The figures are not necessarily to scale. Certain features and components herein may be shown exaggerated in scale or in somewhat schematic form and some details of conventional elements may not be shown in interest of clarity and conciseness.

In the following discussion and in the claims, the terms “including” and “comprising” are used in an open-ended fashion, and thus should be interpreted to mean “including, but not limited to . . . .” As used herein, the terms “approximately,” “about,” “substantially,” and the like mean within 10% (i.e., plus or minus 10%) of the recited value. Thus, for example, a recited angle of “about 80 degrees” refers to an angle ranging from 72 degrees to 88 degrees.

Unless the context dictates the contrary, all ranges set forth herein should be interpreted as being inclusive of their endpoints, and open-ended ranges should be interpreted to include only commercially practical values. Similarly, all lists of values should be considered as inclusive of intermediate values unless the context indicates the contrary.

Disclosed herein are chemoenzymatic methods for the production of carboxylated compounds. In an aspect, the methods of this disclosure involve the reaction of an organic compound in the presence of carbon dioxide and a biocatalyst, such as a carboxylase, under conditions suitable to produce a carboxylic acid intermediate. In some aspects, the method of the present disclosure produce Cy compounds from Cx compounds where y is greater than x.

Carboxylases (sensu stricto, EC 6.4.1.2) are enzymes that catalyze the incorporation of a CO₂ molecule (or a carbonate) into an organic substrate. Hereinafter this is referred to as “Stage 1” of the method.

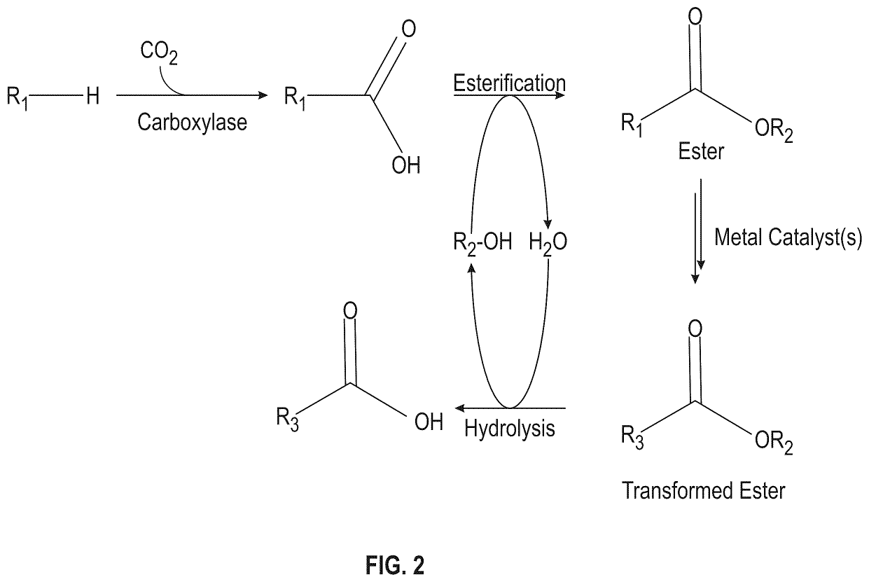

The carboxylic acid intermediate may be further contacted with an alcohol in the presence of one or more metal catalysts under conditions suitable for esterification of the carboxylic acid. This reaction is depicted schematically in FIG. 2.

With reference to FIG. 2, R¹—H represents a natural or non-natural carboxylase substrate where R¹ can be any functional group, R² may be an alkyl chain with a length of C₁-C₆; R³ can be any group derivable from R¹ through any series of dehydration, hydrogenation, reduction, and/or oxidation reactions. Hereinafter this is referred to as “Stage 2” of the method. It is to be understood that the method described may be numerically ordered in stages (i.e., Stage 1, Stage 2) for ease of reference, however, it is not intended to limit the performance of the activities in each stage to a particular order. For example, one or more activities described for a particular stage may be carried out concurrently with one or more activities of another stage whether that “another stage” is designated numerically as being subsequent to or prior to the “particular stage.” Such modifications in terms of the timing of the activities performed in any particular stage may be made by one of ordinary skill in the art with the benefits of the present disclosure.

Generally, R¹, R², or R³ can each independently be an organyl group; alternatively, a C₁ to C₃₀ organyl group; alternatively, a C₁ to C₂₀ organyl group; alternatively, a C₁ to C₁₅ organyl group; alternatively, a C₁ to C₁₀ organyl group; alternatively, a C₁ to C₅ organyl group alternatively, a C₁ to C₃₀ hydrocarbyl group; alternatively, a C₁ to C₂₀ hydrocarbyl group; alternatively, a C₁ to C₁₅ hydrocarbyl group; alternatively, a C₁ to C₁₀ hydrocarbyl group; or alternatively, a C₁ to C₅ hydrocarbyl group; alternatively, C₃ to C₃₀ aromatic group; alternatively, a C₃ to C₂₀ aromatic group; alternatively, a C₃ to C₁₅ aromatic group; or alternatively, a C₃ to C₁₀ aromatic group.

In an aspect, R¹, R², or R³ can each independently be a C₁ to C₃₀ alkyl group, a C₄ to C₃₀ cycloalkyl group, a C₄ to C₃₀ substituted cycloalkyl group, a C₃ to C₃₀ aliphatic heterocyclic group, a C₃ to C₃₀ substituted aliphatic heterocyclic group, a C₆ to C₃₀ aryl group, a C₆ to C₃₀ substituted aryl group, a C₃ to C₃₀ heteroaryl group, or a C₃ to C₃₀ substituted heteroaryl group.

In an aspect, R¹, R², or R³ can each independently be a methyl group, an ethyl group, a propyl group, a butyl group, a pentyl group, a hexyl group, a heptyl group, an octyl group, a nonyl group, a decyl group, a undecyl group, a dodecyl group, a tridecyl group, a tetradecyl group, a pentadecyl group, a hexadecyl group, a heptadecyl group, an octadecyl group, or a nonadecyl group; a halogen or a hydrocarboxy group, a hydrocarboxy group, a cyclobutyl group, a substituted cyclobutyl group, a cyclopentyl group, a substituted cyclopentyl group, a cyclohexyl group, a substituted cyclohexyl group, a cycloheptyl group, a substituted cycloheptyl group, a cyclooctyl group, or a substituted cyclooctyl group.

The term “organyl group” is used herein in accordance with the definition specified by IUPAC: an organic substituent group, regardless of functional type, having one free valence at a carbon atom. Similarly, an “organylene group” refers to an organic group, regardless of functional type, derived by removing two hydrogen atoms from an organic compound, either two hydrogen atoms from one carbon atom or one hydrogen atom from each of two different carbon atoms. An “organic group” refers to a generalized group formed by removing one or more hydrogen atoms from carbon atoms of an organic compound. Thus, an “organyl group,” an “organylene group,” and an “organic group” can contain organic functional group(s) and/or atom(s) other than carbon and hydrogen, that is, an organic group can comprise functional groups and/or atoms in addition to carbon and hydrogen. For instance, non-limiting examples of atoms other than carbon and hydrogen include halogens, oxygen, nitrogen, phosphorus, and the like. Non-limiting examples of functional groups include ethers, aldehydes, ketones, esters, sulfides, amines, phosphines, and so forth. In one aspect, the hydrogen atom(s) removed to form the “organyl group,” “organylene group,” or “organic group” may be attached to a carbon atom belonging to a functional group, for example, an acyl group (—C(O)R), a formyl group (—C(O)H), a carboxy group (—C(O)OH), a hydrocarboxycarbonyl group (—C(O)OR), a cyano group (—C≡N), a carbamoyl group (—C(O)NH₂), an N-hydrocarbylcarbamoyl group (—C(O)NHR), or N,N′-dihydrocarbylcarbamoyl group (—C(O)NR₂), among other possibilities. In another aspect, the hydrogen atom(s) removed to form the “organyl group,” “organylene group,” or “organic group” may be attached to a carbon atom not belonging to, and remote from, a functional group, for example, —CH₂C(O)CH₃, —CH₂NR₂, and the like. An “organyl group,” “organylene group,” or “organic group” may be aliphatic, inclusive of being cyclic or acyclic, or may be aromatic. “Organyl groups,” “organylene groups,” and “organic groups” also encompass heteroatom-containing rings, heteroatom-containing ring systems, heteroaromatic rings, and heteroaromatic ring systems. “Organyl groups,” “organylene groups,” and “organic groups” may be linear or branched unless otherwise specified. Finally, it is noted that the “organyl group,” “organylene group,” or “organic group” definitions include “hydrocarbyl group,” “hydrocarbylene group,” “hydrocarbon group,” respectively, and “alkyl group,” “alkylene group,” and “alkane group,” respectively, as members.

For the purposes of this application, the term or variations of the term “organyl group consisting of inert functional groups” refers to an organyl group wherein the organic functional group(s) and/or atom(s) other than carbon and hydrogen present in the functional group are restricted to those functional group(s) and/or atom(s) other than carbon and hydrogen which do not complex with a metal compound and/or are inert under the process conditions defined herein. Thus, the term or variation of the term “organyl group consisting of inert functional groups” further defines the particular organyl groups that can be present within the organyl group consisting of inert functional groups. Additionally, the term “organyl group consisting of inert functional groups” can refer to the presence of one or more inert functional groups within the organyl group. The term or variation of the term “organyl group consisting of inert functional groups” definition includes the hydrocarbyl group as a member (among other groups). Similarly, an “organylene group consisting of inert functional groups” refers to an organic group formed by removing two hydrogen atoms from one or two carbon atoms of an organic compound consisting of inert functional groups and an “organic group consisting of inert functional groups” refers to a generalized organic group consisting of inert functional groups formed by removing one or more hydrogen atoms from one or more carbon atoms of an organic compound consisting of inert functional groups.

The term “hydrocarbon” whenever used in this specification and claims refers to a compound containing only carbon and hydrogen. Other identifiers can be utilized to indicate the presence of particular groups in the hydrocarbon (e.g. halogenated hydrocarbon indicates that the presence of one or more halogen atoms replacing an equivalent number of hydrogen atoms in the hydrocarbon). The term “hydrocarbyl group” is used herein in accordance with the definition specified by IUPAC: a univalent group formed by removing a hydrogen atom from a hydrocarbon. Non-limiting examples of hydrocarbyl groups include ethyl, phenyl, tolyl, propenyl, and the like. Similarly, a “hydrocarbylene group” refers to a group formed by removing two hydrogen atoms from a hydrocarbon, either two hydrogen atoms from one carbon atom or one hydrogen atom from each of two different carbon atoms. Therefore, in accordance with the terminology used herein, a “hydrocarbon group” refers to a generalized group formed by removing one or more hydrogen atoms (as necessary for the particular group) from a hydrocarbon. A “hydrocarbyl group,” “hydrocarbylene group,” and “hydrocarbon group” can be acyclic or cyclic groups, and/or may be linear or branched. A “hydrocarbyl group,” “hydrocarbylene group,” and “hydrocarbon group” can include rings, ring systems, aromatic rings, and aromatic ring systems, which contain only carbon and hydrogen. “Hydrocarbyl groups,” “hydrocarbylene groups,” and “hydrocarbon groups” include, by way of example, aryl, arylene, arene, alkyl, alkylene, alkane, cycloalkyl, cycloalkylene, cycloalkane, aralkyl, aralkylene, and aralkane groups, among other groups, as members.

The term “alkane” whenever used in this specification and claims refers to a saturated hydrocarbon compound. Other identifiers can be utilized to indicate the presence of particular groups in the alkane (e.g. halogenated alkane indicates that the presence of one or more halogen atoms replacing an equivalent number of hydrogen atoms in the alkane). The term “alkyl group” is used herein in accordance with the definition specified by IUPAC: a univalent group formed by removing a hydrogen atom from an alkane. Similarly, an “alkylene group” refers to a group formed by removing two hydrogen atoms from an alkane (either two hydrogen atoms from one carbon atom or one hydrogen atom from two different carbon atoms). An “alkane group” is a general term that refers to a group formed by removing one or more hydrogen atoms (as necessary for the particular group) from an alkane. An “alkyl group,” “alkylene group,” and “alkane group” can be acyclic or cyclic groups, and/or may be linear or branched unless otherwise specified. Primary, secondary, and tertiary alkyl group are derived by removal of a hydrogen atom from a primary, secondary, tertiary carbon atom, respectively, of an alkane. The n-alkyl group may be derived by removal of a hydrogen atom from a terminal carbon atom of a linear alkane. The groups RCH₂ (R≠H), R₂CH (R≠H), and R₃C (R≠H) are primary, secondary, and tertiary alkyl groups, respectively.

A cycloalkane is a saturated cyclic hydrocarbon, with or without side chains, for example, cyclobutane. Unsaturated cyclic hydrocarbons having one or more endocyclic double or one triple bond are called cycloalkenes and cycloalkynes, respectively. Cycloalkenes and cycloalkynes having only one, only two, only three, etc., endocyclic double or triple bonds, respectively, can be identified by use of the term “mono,” “di,” “tri, etc., within the name of the cycloalkene or cycloalkyne. Cycloalkenes and cycloalkynes can further identify the position of the endocyclic double or triple bonds.

A “cycloalkyl group” is a univalent group derived by removing a hydrogen atom from a ring carbon atom of a cycloalkane.

An aliphatic compound is an acyclic or cyclic, saturated or unsaturated carbon compound, excluding aromatic compounds. Thus, an aliphatic compound is an acyclic or cyclic, saturated or unsaturated carbon compound, excluding aromatic compounds; that is, an aliphatic compound is a non-aromatic organic compound. An “aliphatic group” is a generalized group formed by removing one or more hydrogen atoms (as necessary for the particular group) from the carbon atom of an aliphatic compound. Thus, an aliphatic compound is an acyclic or cyclic, saturated or unsaturated carbon compound, excluding aromatic compounds. That is, an aliphatic compound is a non-aromatic organic compound. Aliphatic compounds and therefore aliphatic groups may contain organic functional group(s) and/or atom(s) other than carbon and hydrogen.

An aromatic compound is a compound containing a cyclically conjugated double bond system that follows the Huckel (4n+2) rule and contains (4n+2) pi-electrons, where n is an integer from 1 to 5. Aromatic compounds include “arenes” (hydrocarbon aromatic compounds) and “heteroarenes,” also termed “hetarenes” (heteroaromatic compounds formally derived from arenes by replacement of one or more methine (−C═) carbon atoms of the cyclically conjugated double bond system with a trivalent or divalent heteroatoms, in such a way as to maintain the continuous pi-electron system characteristic of an aromatic system and a number of out-of-plane pi-electrons corresponding to the Huckel rule (4n+2). While arene compounds and heteroarene compounds are mutually exclusive members of the group of aromatic compounds, a compound that has both an arene group and a heteroarene group are generally considered a heteroarene compound. Aromatic compounds, arenes, and heteroarenes can be monocyclic (e.g., benzene, toluene, furan, pyridine, methylpyridine) or polycyclic unless otherwise specified. Polycyclic aromatic compounds, arenes, and heteroarenes, include, unless otherwise specified, compounds wherein the aromatic rings can be fused (e.g., naphthalene, benzofuran, and indole), compounds where the aromatic groups can be separate and joined by a bond (e.g., biphenyl or 4-phenylpyridine), or compounds where the aromatic groups are joined by a group containing linking atoms (e.g., carbon-the methylene group in diphenylmethane; oxygen-diphenyl ether; nitrogen-triphenyl amine; among others linking groups). As disclosed herein, the term “substituted” can be used to describe an aromatic group, arene, or heteroarene wherein a non-hydrogen moiety formally replaces a hydrogen in the compound, and is intended to be nonlimiting.

An “aromatic group” refers to a generalized group formed by removing one or more hydrogen atoms (as necessary for the particular group and at least one of which is an aromatic ring carbon atom) from an aromatic compound. For a univalent “aromatic group,” the removed hydrogen atom must be from an aromatic ring carbon. For an “aromatic group” formed by removing more than one hydrogen atom from an aromatic compound, at least one hydrogen atom must be from an aromatic hydrocarbon ring carbon. Additionally, an “aromatic group” may have hydrogen atoms removed from the same ring of an aromatic ring or ring system (e.g., phen-1,4-ylene, pyridin-2,3-ylene, naphth-1,2-ylene, and benzofuran-2,3-ylene), hydrogen atoms removed from two different rings of a ring system (e.g., naphth-1,8-ylene and benzofuran-2,7-ylene), or hydrogen atoms removed from two isolated aromatic rings or ring systems (e.g., bis(phen-4-ylene)methane).

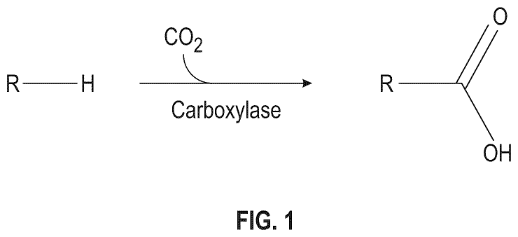

In an aspect, a substrate, generally described in FIG. 1 as R¹—H may be contacted with a biocatalyst capable of introducing carbon (e.g., from CO₂ or CO₃⁻) to the substrate resulting in the formation of a carboxylic acid. Any biocatalyst capable of performing this function may be employed. In an aspect, the biocatalyst is a carboxylase. Carboxylases are enzymes that catalyze the incorporation of a CO₂ molecule into an organic substrate. Although fixation of CO₂ in an organic substrate is common among all carboxylating enzymes, the underlying mechanisms of the carboxylation reactions differ in essential ways with respect to cosubstrate, cofactor, or metal requirements.

Nonlimiting examples of carboxylases suitable for use in the present in the present disclosure include thiamine pyrophosphate (TPP)-dependent decarboxylases, non-TPP dependent decarboxylases, carboxylases, and certain oxidases. While carboxylases rely on ATP cofactors to overcome the energetic requirements for carboxylation, decarboxylases typically function reversibly and can perform carboxylation reactions if exposed to high concentrations of CO₂.

In alternative aspects, the biocatalyst comprises a decarboxylase. Once regarded as irreversible due to CO₂ evolution in the biologically-relevant reaction, decarboxylase reactions have proved reversible. In an aspect, the decarboxylase comprises non-TPP dependent decarboxylases as they do not contain cofactors such as TPP, adenosine triphosphate (ATP), nicotinamide adenine dinucleotide phosphate NAD(P)+/nicotinamide adenine dinucleotide diphosphate NAD(P)H that must be added to the reaction solution, resulting in dramatically increased production costs.

Nonlimiting examples of cofactor-free decarboxylases suitable for use in the present disclosure include 4-hydroxybenzoate decarboxylases, 3,4-dihydroxybenzoate decarboxylases, 2,6-dihydroxybenzoate decarboxylases,-γ-resorcyclate decarboxylase, 2,3-dihydroxybenzoate decarboxylases, 4,5-dihydroxyphthalate decarboxylases, hydroxycinnamate decarboxylases, gallic acid decarboxylases, ferulate decarboxylases, p-coumarate decarboxylases, pyrrole-2-carboxylate decarboxylases, indole-3-carboxylate decarboxylases, orotidine 5′-monophosphate decarboxylases, arylmalonate decarboxylases, acetoacetate decarboxylases, acetolactate decarboxylases.

Nonlimiting examples of suitable TPP-dependent decarboxylases include pyruvate decarboxylase, benzoylformate decarboxylase, formolase, acetolactate synthase, and the α-ketoglutarate dehydrogenase E1 component.

In some aspect, Stage 1 comprises the utilization of one or more oxidases in conjunction with or in lieu of the carboxylase. Nonlimiting examples of oxidases suitable for this disclosure include oxalate oxidase, lactate 2-monooxygenase, pyruvate oxidase, 2-oxoglutarate dioxygenase or combinations thereof.

In an aspect, the decarboxylase comprises pyruvate decarboxylase. Pyruvate decarboxylase (PDC, E.C. 4.1.1.1, also known as 2-oxo-acid decarboxylase, alpha-ketoacid decarboxylase, and pyruvic decarboxylase) is a homotetrameric enzyme that catalyzes the decarboxylation of pyruvic acid to produce acetaldehyde using magnesium and a TPP cofactor. PDC is found in the cytoplasm of prokaryotes, and in the cytoplasm and mitochondria of eukaryotes. In yeast, this enzyme is a key player in the fermentation process that produces ethanol. A homolog of the yeast PDC is also present in filamentous fungi, e.g., Aspergillus spp., but less is known about its exact reaction mechanism.

In an aspect, a PDC suitable for use in the present disclosure may be sourced from microbes with high specific activity on pyruvate including those sourced from Acetobacter pasteurianus PDC, Zymobacter palmae PDC (ZpPDC), Zymomonas mobilis PDC (ZmPDC), Saccharomyces cerevisiae PDC, or other sources mentioned above.

In an aspect, the carboxylase comprises benzoylformate decarboxylase. benzoylformate decarboxylase (BFDC) is a thiamin diphosphate (ThDP)-dependent enzyme that catalyzes the nonoxidative decarboxylation of benzoylformate generating benzaldehyde and carbon dioxide. Originally isolated from Pseudomonas putida, BFDC was found to be the penultimate enzyme in the mandelate pathway, a secondary metabolic pathway that allows various pseudomonads to grow using R-mandelate as their sole source of carbon.

A reaction for formation of a carboxylic acid from a natural or unnatural substrate may be carried out using one or more of the following parameters: substrate in an amount ranging from about 0.1 equivalents to about 100 equivalents or 1 equivalent to about 75 equivalents or about 10 equivalents to about 50 equivalents; a biocatalyst (e.g., PDC) in an amount of from about 10 nanograms/liter (ng/L) to about 10 milligrams/liter (mg/L) or about 100 micrograms/L to about 5 mg/L or about 500 micrograms/L to about 2 mg/L; a cofactor (e.g., TPP) in an amount ranging from about an amount ranging from about 0.1 equivalents to about 100 equivalents or 1 equivalent to about 75 equivalents or about 10 equivalents to about 50 equivalents; a carbon source (e.g., CO₂) present in an amount of from about 0.1 mM to about 1 M or about 1 mM to about 0.5 M or about 10 mM to about 0.1 M; a pH of from about 5 to about 11 or about 6 to about 10 or about 7 to about 9; a reaction media comprising an aqueous buffer; a reaction temperature of from about 0° C. to about 40° C. or from about 5° C. to about 30° C. or about 10° C. to about 25° C.; and a reaction time of from about 0.5 hours to about 72 hours or about 5 hours to about 48 hours or about 12 hours to about 24 hours.

The product mixture may contain carboxylic acid in a yield ranging from about 30% to about 95% based on the total amount of products or about 40% to about 90% or about 50% to about 85% and a purity ranging from about 60% to about 99% or about 70% to about 95% or about 80% to about 90%.

In some aspects, the carboxylic acid is converted to a first ester. This reaction is depicted in FIG. 2. With reference to FIG. 2, the carboxylic acid is reacted with an alcohol in the presence of an alcohol and an acid/base catalyst under conditions suitable for the formation of a first ester. Nonlimiting examples of acid/base catalysts suitable for use in the present disclosure include without limitation hydrochloric acid, sulfuric acid, formic acid, sodium hydroxide and urea. Reaction conditions for formation of the first ester may include one or more of the following: an amount of carboxylic acid ranging from about 0.1 equivalents to about 100 equivalents or 1 equivalent to about 75 equivalents or about 10 equivalents to about 50 equivalents; an amount of alcohol ranging from about 0.1 equivalents to about 100 equivalents or 1 equivalent to about 75 equivalents or about 10 equivalents to about 50 equivalents; an amount of acid/base catalyst ranging from about ranging from about 0.1 equivalents to about 100 equivalents or 1 equivalent to about 75 equivalents or about 10 equivalents to about 50 equivalents; a reaction temperature of from about 25° C. to about 100° C. or from about 30° C. to about 90° C. or about 30° C. to about 80° C.; and a reaction time of from about 0.5 hours to about 72 hours or about 5 hours to about 48 hours or about 12 hours to about 24 hours. The carboxylic acid may be used without further purification or may be purified using any suitable methodology to meet one or more user and/or process goals.

In some aspects, the first ester is produced in the presence of a biocatalyst such as a CalB lipase (i.e., Novozymes 435 immobilized CalB). Because esterification using lipases typically requires anhydrous conditions, this reaction may occur in the presence of tert-butyl alcohol, methyl butyrate, or other solvent, potentially in a biphasic system.

an amount of carboxylic acid ranging from about 0.1 equivalents to about 100 equivalents or 1 equivalent to about 75 equivalents or about 10 equivalents to about 50 equivalents; an amount of alcohol ranging from about 0.1 equivalents to about 100 equivalents or 1 equivalent to about 75 equivalents or about 10 equivalents to about 50 equivalents; an amount of biocatalyst ranging from about 10 nanograms/liter (ng/L) to about 10 milligrams/liter (mg/L) or about 100 micrograms/L to about 5 mg/L or about 500 micrograms/L to about 2 mg/L; a reaction media comprising an anhydrous solvent such as tert-butyl alcohol, methyl butyrate; a temperature ranging from about 0° C. to about 40° C. or from about 5° C. to about 30° C. or about 10° C. to about 25° C. and a reaction time of from about 0.5 hours to about 72 hours or about 5 hours to about 48 hours or about 12 hours to about 24 hours. In some aspects, the reaction is carried out in a biphasic solvent system.

The product mixture may contain the first ester in a yield ranging from about 30% to about 95% based on the total amount of products or about 40% to about 90% or about 50% to about 85% and a purity ranging from about 60% to about 99% or about 70% to about 95% or about 80% to about 90%. The first ester may be used without further purification or may be purified using any suitable methodology to meet one or more user and/or process goals.

In some aspects, a Stage II of the presently disclosed methods comprises contacting the first ester with a catalyst under conditions suitable for the formation of one or more desired products. Herein the catalysts are characterized by the presence of a metal and are designated HORD catalysts which refer collectively to hydrogenation, oxidation, reduction and/or dehydration catalysts.

In an aspect, the HORD catalyst comprises a transition-metal oxidation catalyst. In such aspects, the HORD catalyst is a supported transition-metal oxidation catalyst, alternatively a nanoparticle supported transition-metal oxidation catalyst. In an aspect, the support comprises carbon, silica, alumina, titania (TiO₂), zirconia (ZrO₂), a zeolite, or any combination thereof, which contains less than about 1.0 weight percent (wt. %), alternatively less than about 0.1 wt. % or alternatively less than about 0.01 wt. % SiO₂ binders based on the total weight of the support.

Suitable support materials are predominantly mesoporous or macroporous, and substantially free from micropores. For example, the support may comprise less than about 20% micropores. In an aspect, the support is a porous nanoparticle support. As used herein, the term “micropore” refers to pores with diameter <2 nm, as measured by nitrogen adsorption and mercury porosimetry methods and as defined by IUPAC. As used herein, the term “mesopore” refers to pores with diameter from ca. 2 nm to ca. 50 nm, as measured by nitrogen adsorption and mercury porosimetry methods and as defined by IUPAC. As used herein, the term “macropore” refers to pores with diameters larger than 50 nm, as measured by nitrogen adsorption and mercury porosimetry methods and as defined by IUPAC.

In an aspect, the support comprises a mesoporous carbon extrudate having a mean pore diameter ranging from about 10 nm to about 100 nm and a surface area greater than about 20 m² g⁻¹ but less than about 300 m² g⁻¹. Supports suitable for use in the present disclosure may have any suitable shape. For example, the support may be shaped into 0.8-3 mm trilobes, quadralobes, or pellet extrudates. Such shaped supports enable the used of fixed trickle bed reactors to perform the final oxidation step under continuous flow. In one or more aspects, the metal comprises a Group 8 metal (e.g., Re, Os, Ir, Pt, Ru, Rh, Pd, Ag), a 3d transition metal, an early transition metal, or combinations thereof. In an aspect, the HORD comprises gold, Au.

In an aspect, the HORDs comprise platinum and gold and are heterogeneous, solid-phase HORDs. In such aspects, suitable catalyst supports include, without limitation, carbon, surface treated aluminas (such as passivated aluminas or coated aluminas), silicas, titanias, zirconias, zeolites, montmorillonites, and modifications, mixtures or combinations thereof. The catalyst support may be treated to promote the preferential deposition of platinum and gold on the outer surface of the support so as to create a shell type HORD. The platinum and gold-containing compounds that function as a HORD may be produced by any suitable methodology. For example, the platinum and gold-containing HORDs may be produced using deposition procedures such as incipient wetness, ion-exchange and deposition-precipitation.

In other aspects, the HORD comprises metal phases that are monometallic or multimetallic combinations of Cu, Ag, Au, Ni, Pd, Pt, and Ir. The activity, selectivity, and stability of the active phases can be modulated with dopants of early 3d, 4d, and 5d transition metals, or heavy post transition metals such as Sn, Sb, and Bi. In some aspects, metals (e.g., Group 1 metals) are intercalated into the metal lattice to modulate catalyst properties. In an aspect, salt precursors of the active phases are deposited onto a support of the type disclosed herein using any suitable methodology. For example, deposition of the active phases may be carried out using techniques such as incipient wetness impregnation, bulk adsorption impregnation, or deposition precipitation.

In an aspect, the deposited salt precursor of the active phase is then converted to the active phase via Liquid Phase Reduction (LPR) with a suitable salt (e.g., formate salt) at temperatures of less than about 100° C. or via Gas Phase Reduction (GPR) at temperatures ranging from about 200° C. to about 500° C. or alternatively from about 200° C. to about 450° C.

In an aspect, the amount of active phase loaded onto a support of the type disclosed herein is less than about 2.0 weight percent (wt. %), alternatively less than about 1.5 wt. % or alternatively less than about 1.0 wt. % based on the total weight of the HORD. In an aspect, the amount of active phase loaded onto a support of the type disclosed herein is equal to or less than about 0.5 wt. % based on the total weight of the HORD catalyst. In an aspect, the radial distribution of the active phase across the support is anisotropic where the active phase is substantially concentrated in a <500 μm annulus near the surface of the extrudate support in a “core-shell” configuration. A HORD catalyst of the type disclosed herein may be characterized by a productivity for the conversion of aldehyde functionalities to carboxylic acids of equal to or greater than about 0.05 mol acid g⁻¹ active metal h⁻¹ or equal to or greater than about 0.1 mol acid g⁻¹ active metal h⁻¹ at selectivities from about 70% to about 90%, alternatively equal to or greater than about 70%, alternatively equal to or greater than about 80%, alternatively equal to or greater than about 85%, or alternatively equal to or greater than about 90%. In such aspects, the HORD catalyst exhibits conversions of from about 60% to about 95%, alternatively equal to or greater than about 70%, alternatively equal to or greater than about 80%, or alternatively equal to or greater than about 90%. In an aspect, a HORD catalyst of the type disclosed herein may be utilized in a temperature range of from about 40° C. to about 120° C., alternatively form about 40° C. to about 110° C. or alternatively from about 50° C. to about 100° C. at pressures ranging from about 10 bar to about 100 bar, alternatively from about 20 bar to about 100 bar or alternatively from about 20 bar to about 90 bar.

For example, and with reference to FIG. 2, the first ester may be reacted with an alcohol (R³—OH) in the presence of a HORD catalyst under conditions suitable for the formation of a transformed ester. Reaction conditions for preparation of the transformed ester in the presence of a HORD catalyst may include one or more of the following: an amount of first ester ranging from about 0.1 equivalents to about 100 equivalents or 1 equivalent to about 75 equivalents or about 10 equivalents to about 50 equivalents; an amount of alcohol ranging from about 0.1 equivalents to about 100 equivalents or 1 equivalent to about 75 equivalents or about 10 equivalents to about 50 equivalents; an amount of HORD catalyst ranging from about 0.1 equivalents to about 100 equivalents or 1 equivalent to about 75 equivalents or about 10 equivalents to about 50 equivalents; a reaction temperature of from about 25° C. to about 100° C. or from about 30° C. to about 90° C. or about 30° C. to about 80° C.; and a reaction time of from about 0.5 hours to about 72 hours or about 5 hours to about 48 hours or about 12 hours to about 24 hours. The product mixture may contain the transformed ester in a yield ranging from about 30% to about 95% based on the total amount of products or about 40% to about 90% or about 50% to about 85% and a purity ranging from about 60% to about 99% or about 70% to about 95% or about 80% to about 90%.

The transformed ester may be used without further purification or may be purified using any suitable methodology to meet one or more user and/or process goals.

In one or more aspects, the transformed ester may be converted to a carboxylic acid via hydrolysis. With reference to FIG. 2, the transformed ester may be hydrolyzed in the presence of a base to form a second carboxylic acid. Reaction conditions for preparation of the second carboxylic acid may include one or more of the following: an amount of transformed ester ranging from about 0.1 equivalents to about 100 equivalents or 1 equivalent to about 75 equivalents or about 10 equivalents to about 50 equivalents; an amount of base catalyst ranging from about 0.1 equivalents to about 100 equivalents or 1 equivalent to about 75 equivalents or about 10 equivalents to about 50 equivalents; a reaction temperature of from about 25° C. to about 100° C. or from about 30° C. to about 90° C. or about 30° C. to about 80° C.; and a reaction time of from about 0.5 hours to about 72 hours or about 5 hours to about 48 hours or about 12 hours to about 24 hours.

The product mixture may contain the second carboxylic acid in a yield ranging from about 30% to about 95% based on the total amount of products or about 40% to about 90% or about 50% to about 85% and a purity ranging from about 60% to about 99% or about 70% to about 95% or about 80% to about 90%.

The second carboxylic acid may be used without further purification or may be purified using any suitable methodology to meet one or more user and/or process goals.

In an aspect, a biocatalyst of the type disclosed herein is a wild type enzyme, a functional fragment thereof or a functional variant thereof. “Fragment” as used herein is meant to include any amino acid sequence shorter than the full-length biocatalyst (e.g., PDC), but where the fragment maintains a catalytic activity sufficient to meet some user or process goal. Fragments may include a single contiguous sequence identical to a portion of the biocatalyst sequence. Alternatively, the fragment may have or include several different shorter segments where each segment is identical in amino acid sequence to a different portion of the amino acid sequence of the biocatalyst but linked via amino acids differing in sequence from the biocatalyst. Herein, a “functional variant” of the biocatalyst refers to a polypeptide which has at one or more positions of an amino acid insertion, deletion, or substitution, either conservative or non-conservative, and wherein each of these types of changes may occur alone, or in combination with one or more of the others, one or more times in a given sequence but retains catalytic activity.

In the alternative or in combination with the aforementioned mutations, the biocatalyst may be mutated to improve the catalytic activity.

In an aspect, any biocatalyst of the type disclosed herein may be cloned into an appropriate expression vector and used to transform cells of an expression system such as E. coli, Saccharomyces sp., Pichia sp., Aspergillus sp., Trichoderma sp., or Myceliophthora sp. A “vector” is a replicon, such as plasmid, phage, viral construct or cosmid, to which another DNA segment may be attached. Vectors are used to transduce and express a DNA segment in cells. As used herein, the terms “vector” and “construct” may include replicons such as plasmids, phage, viral constructs, cosmids, Bacterial Artificial Chromosomes (BACs), Yeast Artificial Chromosomes (YACs) Human Artificial Chromosomes (HACs) and the like into which one or more AOX gene expression cassettes may be or are ligated. Herein, a cell has been “transformed” by an exogenous or heterologous nucleic acid or vector when such nucleic acid has been introduced inside the cell, for example, as a complex with transfection reagents or packaged in viral particles. The transforming DNA may or may not be integrated (covalently linked) into the genome of the cell.

In an aspect, the gene of a biocatalyst disclosed herein is provided as a recombinant sequence in a vector where the sequence is operatively linked to one or more control or regulatory sequences. “Operatively linked” expression control sequences refers to a linkage in which the expression control sequence is contiguous with the gene of interest to control the gene of interest, as well as expression control sequences that act in trans or at a distance to control the gene of interest.

The term “expression control sequence” or “regulatory sequences” are used interchangeably and are used herein refer to polynucleotide sequences, which are necessary to affect the expression of coding sequences to which they are operatively linked. Expression control sequences are sequences that control the transcription, post-transcriptional events, and translation of nucleic acid sequences. Expression control sequences include appropriate transcription initiation, termination, promoter, and enhancer sequences; efficient RNA processing signals such as splicing and polyadenylation signals; sequences that stabilize cytoplasmic mRNA; sequences that enhance translation efficiency (e.g., ribosome binding sites); sequences that enhance protein stability; and when desired, sequences that enhance protein secretion. The nature of such control sequences differs depending upon the host organism; in prokaryotes, such control sequences generally include promoter, ribosomal binding site, and transcription termination sequence. The term “control sequences” is intended to include, at a minimum, all components whose presence is essential for expression, and can also include additional components whose presence is advantageous, for example, leader sequences and fusion partner sequences.

The term “recombinant host cell” (“expression host cell”, “expression host system”, “expression system” or simply “host cell”), as used herein, is intended to refer to a cell into which a recombinant vector has been introduced. It should be understood that such terms are intended to refer not only to the particular subject cell but to the progeny of such a cell. Because certain modifications may occur in succeeding generations due to either mutation or environmental influences, such progeny may not, in fact, be identical to the parent cell, but are still included within the scope of the term “host cell” as used herein. A recombinant host cell may be an isolated cell or cell line grown in culture or may be a cell which resides in a living tissue or organism.

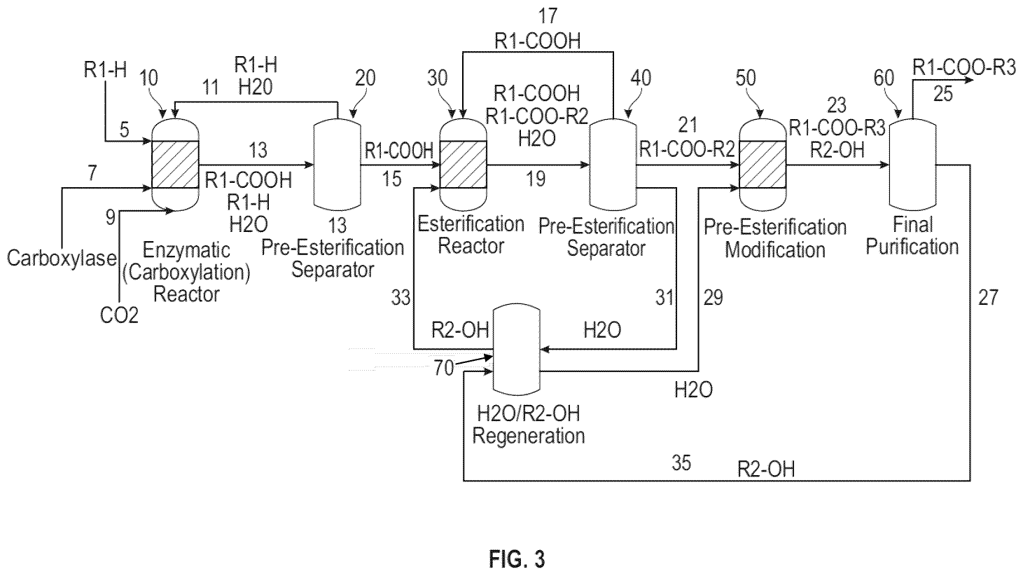

In one or more aspects, chemoenzymatic carboxylation is carried out in a reactor system. FIG. 3 is a process flow diagram of a reactor system 100, depicting an aspect of the presently disclosed methods.

Referring to FIG. 3, an enzymatic carboxylation reactor 10 could be a sparged bubble column (as shown), an air lift column or a stirred sparged bioreactor. Operating ranges for the enzymatic carboxylation reactor 10 include temperatures of from about 20° C. to about 100° C. or from about 30° C. to about 90° C. or from about 30° C. to about 60° C. and pressures ranging from about 1 bar to about 20 bar or from about 1 bar to about 15 bar or from about 1 bar to about 10 bar.

In the enzymatic carboxylation reactor 10 the reagents R¹—H, carboxylase and a carbon source (e.g., CO₂) are introduced via conduits 5, 7 and 9 respectively. Carbon dioxide may be introduced to the enzymatic carboxylation reactor 10 to drive carboxylation. This may be accomplished by either sparging high pressure CO₂ from the bottom of the enzymatic carboxylation reactor 10, or the addition of supercritical CO₂ from the bottom of the enzymatic carboxylation reactor 10, or the addition of supercritical CO₂ in a higher-pressure reactor configuration (e.g., greater than about 1070 PSIG).

In the enzymatic carboxylation reaction 10, R¹—H undergoes carboxylation to form R¹—COOH. In one or more aspects, the enzymatic carboxylation reactor 10 product is conveyed via conduit 13 to a post esterification separation unit 20.

Enzyme can be recovered from the reactor effluent of the enzymatic carboxylation reactor 10 using any suitable separation unit 20 or methodology such as a tangential flow filtration (TFF) in the case of a homogenous reactor product or via liquid-liquid centrifugation (LCC) in the event of a heterogeneous reactor product, or via liquid-liquid centrifugation (LLC) in the event of a heterogeneous organic/aqueous product. In one or more aspects, the aqueous, enzyme-rich phase may be recycled back to the enzymatic carboxylation reactor 10. The extent of carboxylic acid formation in esterification reactors is equilibrium-limited in the presence of water; as such water may be removed from the feeds. In aspects, the enzymatic carboxylation reactor 10 naturally forms an aqueous/organic phase partition, in such aspects water removal can be accomplished via gravity settling or a liquid-liquid centrifuge. In other aspects, the enzymatic carboxylation reactor 10 product is a homogenous mixture of carboxylic acid and water, water removal may be accomplished utilizing methodologies such as fractional distillation and/or molecular sieve drying. The resulting material comprising the carboxylic acid to esterification unit 30 via conduit 15.

In esterification unit 30, esterification of R¹—COOH to R¹—COO—R² may be achieved using any suitable catalyst. For example, esterification reactor 30 contains a lipase such as a CalB lipase (e.g., NOVOZYMES 435 immobilized CalB). Because esterification using lipases typically requires anhydrous conditions, this reaction may occur in the presence of tert-butyl alcohol, methyl butyrate, or other solvent potentially in a biphasic system with the water produced in the reaction. In some aspects, disposed within the esterification reactor 30 is an acid catalyst such as sulfuric acid. In some aspects, the esterification reactor 30 allows for the reaction to occur via reactive distillation for example under low-temperature vacuum conditions.

In some aspects, a post-esterification separator 40 may be disposed downstream of the esterification reactor 30. In such aspects, the product of the esterification reactor 30 may be conveyed via conduit 19 to the post-esterification separator 40. The post esterification separator 40 may be used to (i) purify ester product. (ii) remove water by-product and (iii) return unreacted R¹—COOH to the esterification reactor, for example via recycle conduit 17. The post-esterification separator 40 may include a distillation column, vacuum distillation, extractive distillation, liquid-liquid separation, a nanofiltration unit or combinations thereof.

In one or more aspects, the esterified product can be used with or without further processing. In other aspects, the R¹—COO—R² is further reacted to produce the end product, R³—COOH. For example, the ester may be introduced to a post esterification modification reactor 50. In some aspects, disposed within the post esterification modification reactor 50 is a metal catalyst. The metal catalyst may be used to further transform the esterified compound. Transformations may include hydrogenation/reduction, dehydration, and/or oxidation.

For hydrogenation, heterogenous monometallic or bimetallic catalysts of the chemical elements Ru, Pd, Pt, Ni, Cu, and Co can be utilized. In some aspects, these catalysts are supported on materials such as carbon, silica, hydrotalcite or titania. With regard to the hydrogenation catalysts, reducing agents like NaBH₄ and formaldehyde may be used to stabilize the cluster size on the catalyst surface.

For dehydration, heterogeneous catalysts such as phosphate metal oxides, Amberlyst-based, alumina, niobia, titania, zirconia can be utilized. In such reactions, suitable solvents include water, alcohols such as methanol, butanol and water and ionic liquids.

For oxidation, catalysts such as supported noble metals (e.g., gold on carbon (Au/C) can be utilized. In an aspect, an oxidation catalyst suitable for use in the present disclosure comprises a carbon support with a porosity between 2 nm-50 nm where the metal cluster sizes ranging from 2 nm to 10 nm are placed.

For hydrolysis, the ester is reacted with water, optionally supplemented with a lipase or catalyzed by a strong base. In some aspects, hydrolysis of the esters is carried out in the presence of a homogeneous or heterogenous catalysts. Nonlimiting aspects of suitable catalysts include acidic heterogeneous catalysts like zeolite molecular sieves such as SBA molecular sieves, HY zeolites, HBeta zeolites, HZSM-5 or heteropoly acids. The catalyst acidity can be calculated to values between 1.0 mmol/g to 2.2 mmol/g. The selectivity of the process is mainly driven by the tailoring of the surface acidity of the heterogeneous catalyst. In addition, the porosity of the catalyst and the catalyst support render possible high conversion rates. In an aspect, the porosity of the metal catalyst ranges from about 2 nm to about 50 nm. In another aspect, hydrolysis can be catalyzed by strong acids such as hydrofluoric acid, hydrochloric acid, and sulfuric acid. The catalytic reaction is preferably conducted in water, for all hydrolysis processes.

After final chemical modification, the end product R³—COOH can be further refined to the desired purity in a purification unit 60. The purification unit 60 may contain one or more mechanisms for liquid-liquid extraction, fractional distillation, extractive distillation, simulated moving bed chromatography, crystallization, Nutsche filtration or combinations thereof.

With regard to FIG. 3, in conjunction with any of the aforementioned processes, water may R²—OH be generated and consumed to drive the reaction forward, effectively functioning as process catalysts. These species can be re-used by purifying them when they are generated in regenerator 70, and re-routing the purified intermediates back to where they are consumed in the reaction. The regeneration can be achieved by either flash distillation, fractional distillation or liquid-liquid extraction.

Although FIG. 3 is at a process flow diagram level of detail, not all process interconnections are shown such as spillbacks, block and bleeds, recycle lines, control valves, cooling/heating elements, pumps, intermediate tankage, antifoam, etc.

The present disclosure provides for a chemoenzymatic process of generating molecules with increased carbon number given a carbon supply (e.g., CO₂). Enzymes capable of incorporating carbon dioxide into a molecule may be disposed in a pressurized, cooled vessel containing carbon dioxide in gaseous, liquid (e.g., concentrated carbonate/bicarbonate), or supercritical state to enhance interaction with the enzyme. Products, which are typically acids, are esterified enzymatically (e.g., with lipases such as CalB) or chemically, then optionally further reacted to subsequent products before hydrolysis to the acid form.

The compositions and methods disclosed herein will generate carboxylated products at high purity using an in vitro system. Use of highly specific enzymes as catalysts eliminates or reduces the presence of additional compounds and materials, facilitating separation and purification of the product and leading to lower production costs compared to traditional chemical synthesis using petroleum-based feedstocks. In an aspect, the compositions, methods and processes disclosed herein result in products having a final product purity of equal to or greater than about 80%, 81%, 82%, 83%, 84%, 85%, 86%, 87%, 88%, 89%, 90%, 91%, 92%, 93%, 94%, 95%, 96%, 97%, 98% or 99%. Further the chemoenzymatic methods disclosed herein result in the direct carboxylation with carbon dioxide followed by selective partial hydrogenations at high space time yields representing the lowest feedstock cost basis pathway to highly pure carboxylated molecules. Further advantages of the present disclosure are attributable to the use of the enzymatic catalysts which function at lower temperatures, and lower operating pressures (100 psi in the enzymatic process) will lead to an inherently safer process. The use of carbon dioxide as a substrate for chemical production results in a process that reduces atmospheric carbon dioxide to combat anthropogenic climate change.

### ADDITIONAL DISCLOSURE

The following are non-limiting, specific aspects in accordance with the present disclosure:

A first aspect which is a method of increasing the carbon content of an organic compound comprising carboxylating an organic compound characterized by the general formula R¹—H and carbon dioxide in the presence of a biocatalyst under conditions suitable for the formation of a carboxylic acid characterized by the general formula R¹—OOH, wherein R¹ is a C₁ to C₃₀ organyl group; a C₁ to C₃₀ hydrocarbyl group, a C₃ to C₃₀ aromatic group; a C₁ to C₃₀ alkyl group, a C₄ to C₃₀ cycloalkyl group, a C₄ to C₃₀ substituted cycloalkyl group, a C₃ to C₃₀ aliphatic heterocyclic group, a C₃ to C₃₀ substituted aliphatic heterocyclic group, a C₆ to C₃₀ aryl group, a C₆ to C₃₀ substituted aryl group, a C₃ to C₃₀ heteroaryl group, or a C₃ to C₃₀ substituted heteroaryl group or combinations thereof.

A second aspect which is the method of the first aspect wherein R¹ is a C₁ to C₁₀ alkyl group.

A third aspect which is the method of any of the first through second aspects wherein R¹ is a methyl group, an ethyl group, a propyl group, a butyl group, a pentyl group, a hexyl group, a heptyl group, an octyl group, a nonyl group, a decyl group, a undecyl group, a dodecyl group, a tridecyl group, a tetradecyl group, a pentadecyl group, a hexadecyl group, a heptadecyl group, an octadecyl group, or a nonadecyl group; a halogen or a hydrocarboxy group; alternatively, a halogen; or alternatively, a hydrocarboxy group, a cyclobutyl group, a substituted cyclobutyl group, a cyclopentyl group, a substituted cyclopentyl group, a cyclohexyl group, a substituted cyclohexyl group, a cycloheptyl group, a substituted cycloheptyl group, a cyclooctyl group, or a substituted cyclooctyl group.

A fourth aspect which is the method of any of the first through third aspects wherein R¹ is a methyl group, an ethyl group, a propyl group, a butyl group, or a pentyl group.

A fifth aspect which is the method of any of the first through fourth aspects wherein R¹ is a methyl group, an ethyl group, or a propyl group.

A sixth aspect which is the method of any of the first through fifth aspects wherein the carboxylic acid is formed in a yield of from about 30% to about 95%.

A seventh aspect which is the method of any of the first through sixth aspects wherein the biocatalyst comprises a cofactor-free decarboxylase.

An eighth aspect which is the method of any of the first through seventh aspects wherein the biocatalyst comprises 4-hydroxybenzoate decarboxylases, 3,4-dihydroxybenzoate decarboxylases, 2,6-dihydroxybenzoate decarboxylases,-γ-resorcyclate decarboxylase, 2,3-dihydroxybenzoate decarboxylases, 4,5-dihydroxyphthalate decarboxylases, hydroxycinnamate decarboxylases, gallic acid decarboxylases, ferulate decarboxylases, p-coumarate decarboxylases, pyrrole-2-carboxylate decarboxylases, indole-3-carboxylate decarboxylases, orotidine 5′-monophosphate decarboxylases, arylmalonate decarboxylases, acetoacetate decarboxylases, acetolactate decarboxylases or combinations thereof.

A ninth aspect which is the method of any the first through eighth aspects wherein the biocatalyst comprises a thiamine pyrophosphate-dependent decarboxylase.

A tenth aspect which is the method of the ninth aspect wherein the biocatalyst comprises pyruvate decarboxylase, benzoylformate decarboxylase, formolase, acetolactate synthase, α-ketoglutarate dehydrogenase E1 component or combinations thereof.

An eleventh aspect which is the method of any of the first through tenth aspects further comprising contacting the carboxylic acid with an alcohol characterized by the general formula R²—OH in the presence of a catalyst under conditions suitable for the formation of a first ester characterized by the general formula R¹COOR².

A twelfth aspect which is the method of the eleventh aspect wherein R¹ or R² are each independently a C₁ to C₃₀ organyl group; a C₁ to C₃₀ hydrocarbyl group, a C₃ to C₃₀ aromatic group; a C₁ to C₃₀ alkyl group, a C₄ to C₃₀ cycloalkyl group, a C₄ to C₃₀ substituted cycloalkyl group, a C₃ to C₃₀ aliphatic heterocyclic group, a C₃ to C₃₀ substituted aliphatic heterocyclic group, a C₆ to C₃₀ aryl group, a C₆ to C₃₀ substituted aryl group, a C₃ to C₃₀ heteroaryl group, or a C₃ to C₃₀ substituted heteroaryl group or combinations thereof.

A thirteenth aspect which is the method of any of the eleventh through twelfth aspects wherein the catalyst is an acid catalyst.

A fourteenth aspect which is the method of any of the eleventh through twelfth aspects wherein the catalyst is a base catalyst.

A fifteenth aspect which is the method of any of the first through fifteenth aspects further comprising contacting the ester in the presence of an alcohol characterized by the general formula R³—OH with a metal catalyst under conditions suitable for transesterification of the first ester characterized by the general formula R³COOR² to form a second ester R¹COOR² selected from the group consisting of hydrogenation catalyst, oxidation catalyst, reduction catalyst, dehydration catalyst and combinations thereof.

A sixteenth aspect which is the method of the fifteenth aspects wherein metal catalyst comprises a transition metal and a support.

A seventeenth aspect which is the method of the sixteenth aspect wherein the support carbon, silica, alumina, titania (TiO₂), zirconia (ZrO₂), a zeolite, or any combination thereof.

An eighteenth aspect which is the method of any of the fifteenth through seventeenth aspects wherein the transition metal comprises Re, Os, Ir, Pt, Ru, Rh, Pd, Ag, a 3d transition metal, an early transition metal, or combinations thereof.

A nineteenth aspect which is the method of any of the fifteenth through eighteenth aspects wherein the metal catalyst comprises gold on a carbon support.

A twentieth aspect which is the method of any of the fifteenth through nineteenth aspects further comprising hydrolyzing the second ester to form a second carboxylic acid.

While aspects of the presently disclosed subject matter have been shown and described, modifications thereof can be made by one skilled in the art without departing from the spirit and teachings of the subject matter. The aspects described herein are exemplary only, and are not intended to be limiting. Many variations and modifications of the subject matter disclosed herein are possible and are within the scope of the disclosed subject matter. Where numerical ranges or limitations are expressly stated, such express ranges or limitations should be understood to include iterative ranges or limitations of like magnitude falling within the expressly stated ranges or limitations (e.g., from about 1 to about 10 includes, 2, 3, 4, etc.; greater than 0.10 includes 0.11, 0.12, 0.13, etc.). Use of the term “optionally” with respect to any element of a claim is intended to mean that the subject element is required, or alternatively, is not required. Both alternatives are intended to be within the scope of the claim. Use of broader terms such as comprises, includes, having, etc. should be understood to provide support for narrower terms such as consisting of, consisting essentially of, comprised substantially of, etc.

Accordingly, the scope of protection is not limited by the description set out above but is only limited by the claims which follow, that scope including all equivalents of the subject matter of the claims. Each and every claim is incorporated into the specification as an aspect of the present disclosure. Thus, the claims are a further description and are an addition to the aspects of the presently disclosed subject matter. The discussion of a reference herein is not an admission that it is prior art to the presently disclosed subject matter, especially any reference that may have a publication date after the priority date of this application. The disclosures of all patents, patent applications, and publications cited herein are hereby incorporated by reference, to the extent that they provide exemplary, procedural or other details supplementary to those set forth herein.

What is claimed is:

1. A method of increasing the carbon content of an organic compound comprising:
carboxylating an organic compound characterized by the general formula R¹—H and carbon dioxide in the presence of a biocatalyst under conditions suitable for the formation of a carboxylic acid characterized by the general formula R¹—OOH, wherein R¹ is a C₁ to C₃₀ organyl group; a C₁ to C₃₀ hydrocarbyl group, a C₃ to C₃₀ aromatic group; a C₁ to C₃₀ alkyl group, a C₄ to C₃₀ cycloalkyl group, a C₄ to C₃₀ substituted cycloalkyl group, a C₃ to C₃₀ aliphatic heterocyclic group, a C₃ to C₃₀ substituted aliphatic heterocyclic group, a C₆ to C₃₀ aryl group, a C₆ to C₃₀ substituted aryl group, a C₃ to C₃₀ heteroaryl group, or a C₃ to C₃₀ substituted heteroaryl group or combinations thereof.

2. The method of claim 1, wherein R¹ is a C₁ to C₁ alkyl group.

3. The method of claim 1, wherein R¹ is a methyl group, an ethyl group, a propyl group, a butyl group, a pentyl group, a hexyl group, a heptyl group, an octyl group, a nonyl group, a decyl group, a undecyl group, a dodecyl group, a tridecyl group, a tetradecyl group, a pentadecyl group, a hexadecyl group, a heptadecyl group, an octadecyl group, or a nonadecyl group; a halogen or a hydrocarboxy group; alternatively, a halogen; or alternatively, a hydrocarboxy group, a cyclobutyl group, a substituted cyclobutyl group, a cyclopentyl group, a substituted cyclopentyl group, a cyclohexyl group, a substituted cyclohexyl group, a cycloheptyl group, a substituted cycloheptyl group, a cyclooctyl group, or a substituted cyclooctyl group.

4. The method of claim 2, wherein R¹ is a methyl group, an ethyl group, a propyl group, a butyl group, or a pentyl group.

5. The method of claim 1, wherein R¹ is a methyl group, an ethyl group, or a propyl group.

6. The method of claim 1, wherein the carboxylic acid is formed in a yield of from about 30% to about 95%.

7. The method of claim 1, wherein the biocatalyst comprises a cofactor-free decarboxylase.

8. The method of claim 7, wherein the biocatalyst comprises 4-hydroxybenzoate decarboxylases, 3,4-dihydroxybenzoate decarboxylases, 2,6-dihydroxybenzoate decarboxylases,-γ-resorcyclate decarboxylase, 2,3-dihydroxybenzoate decarboxylases, 4,5-dihydroxyphthalate decarboxylases, hydroxycinnamate decarboxylases, gallic acid decarboxylases, ferulate decarboxylases, p-coumarate decarboxylases, pyrrole-2-carboxylate decarboxylases, indole-3-carboxylate decarboxylases, orotidine 5′-monophosphate decarboxylases, arylmalonate decarboxylases, acetoacetate decarboxylases, acetolactate decarboxylases or combinations thereof.

9. The method of claim 1, wherein the biocatalyst comprises a thiamine pyrophosphate-dependent decarboxylase.

10. The method of claim 9, wherein the biocatalyst comprises pyruvate decarboxylase, benzoylformate decarboxylase, formolase, acetolactate synthase, α-ketoglutarate dehydrogenase E1 component or combinations thereof.

11. The method of claim 1, further comprising contacting the carboxylic acid with an alcohol characterized by the general formula R²—OH in the presence of a catalyst under conditions suitable for the formation of a first ester characterized by the general formula R¹COOR².

12. The method of claim 11, wherein R¹ or R² are each independently a C₁ to C₃₀ organyl group; a C₁ to C₃₀ hydrocarbyl group, a C₃ to C₃₀ aromatic group; a C₁ to C₃₀ alkyl group, a C₄ to C₃₀ cycloalkyl group, a C₄ to C₃₀ substituted cycloalkyl group, a C₃ to C₃₀ aliphatic heterocyclic group, a C₃ to C₃₀ substituted aliphatic heterocyclic group, a C₆ to C₃₀ aryl group, a C₆ to C₃₀ substituted aryl group, a C₃ to C₃₀ heteroaryl group, or a C₃ to C₃₀ substituted heteroaryl group or combinations thereof.

13. The method of claim 11, wherein the catalyst is an acid catalyst.

14. The method of claim 11, wherein the catalyst is a base catalyst.

15. The method of claim 11, further comprising contacting the ester in the presence of an alcohol characterized by the general formula R³—OH with a metal catalyst under conditions suitable for transesterification of the first ester characterized by the general formula R³COOR² to form a second ester R¹COOR selected from the group consisting of hydrogenation catalyst, oxidation catalyst, reduction catalyst, dehydration catalyst and combinations thereof.

16. The method of claim 15, wherein the metal catalyst comprises a transition metal and a support.

17. The method of claim 16, wherein the support carbon, silica, alumina, titania (TiO₂), zirconia (ZrO₂), a zeolite, or any combination thereof.

18. The method of claim 15, wherein the transition metal comprises Re, Os, Ir, Pt, Ru, Rh, Pd, Ag, a 3d transition metal, an early transition metal, or combinations thereof.

19. The method of claim 15, wherein the metal catalyst comprises gold on a carbon support.

20. The method of claim 15, further comprises hydrolyzing the second ester to form a second carboxylic acid.
